(19) United States
(12) Patent Application Publication
(10) Pub. No.: US 2023/0200460 A1
(43) Pub. Date: Jun. 29, 2023

(54) SMART UNDERWEAR SYSTEM FOR VARICOCELE PATIENTS

(71) Applicant: ATATÜRK ÜNIVERSITESI REKTÖRLÜGÜ BILIMSEL ARASTIRMA PROJELERI ( BAP ) KOORDINASYON BIRIMI, Yakutiye/Erzurum (TR)

(72) Inventors: Mete YAGANOGLU, Erzurum (TR); Isa ÖZBEY, Erzurum (TR)

(21) Appl. No.: 17/910,388

(22) PCT Filed: Dec. 27, 2021
(86) PCT No.: PCT/TR2021/051535

§ 371 Date: Sep. 9, 2022

(51) Int. Cl. A41B 9/02; A61B 5/01

(52) U.S. Cl. CPC A41B 9/02; A61B 5/015; A61B 5/1114

(57) ABSTRACT

A smart underwear (7) system that enables the detection of varicocele disease and the follow-up of varicocele patients, characterized by comprising: at least 2 temperature sensors (5) that enable the temperature of the right and left testicles to be measured, and microcontroller (2), for evaluating the data received from the temperature sensors (5) and, when a certain temperature value is exceeded, warning the user to bring his testicles to the appropriate temperature by means of the vibration motor (3), providing information to the user and/or doctor via SMS and e-mail when the testicular temperature exceeds the specified temperature, transferring and monitoring temperature and disease-related data to the cloud service.

### FIELD OF THE INVENTION

The invention relates to the smart underwear system used in the field of urology in the healthcare industry for the detection of varicocele disease and for the follow-up of varicocele patients.

### STATE OF THE ART

Varicocele, the most common and correctable cause of male infertility, is abnormal dilatation of the veins in the pampiniform plexus [1]. The cause of varicocele-induced infertility is not fully understood; however, factors such as temperature increase, increased or decreased blood flow, testicular reflux of renal and adrenal metabolites, hypoxia, hormonal disorder, family history, age, trauma, obesity, phlebitis and long-term survival are thought to play a role [2-5]. The prevalence of varicocele is 15-20% in healthy males. The prevalence of varicocele in males seeking treatment for primary infertility is 35-40%, while varicocele is detected in approximately 80% of those applying for secondary infertility [6,7]. The gold standard method for the diagnosis of a clinically significant varicocele is still a physical examination. Clinical varicoceles are divided into three degrees according to the physical examination. Grade 1 varicocele is small and can only be palpated with valsalva. Grade 2 varicocele is a varicocele that can be palpated at rest. Grade 3 varicocele is a large varicocele that is visible even at rest [8]. The World Health Organization expanded this grading system and added subclinical varicocells that could not be palpated during the valsalva maneuver but could only be detected by ultrasonography (US) [9]. However, clinical examination is subjective, there is a significant difference between the examiners, change in testicular blood flow cannot be detected by physical examination, and physical examination is not sufficient for subclinical varicocele. In addition, physical examination is problematic in obese people, highly scrotal testicles, those with a history of scrotal or inguinal surgery, in the presence of hydrocele, and in postoperative recurrent varicocele. Varicocele diagnosis may be necessary with imaging methods in these cases [10]. However, some authors insist that there is no gold standard method to diagnose subclinical varicocele [11]. In addition to the fact that imaging methods are important for the diagnosis of varicocele, they also have a role in examining the effect of varicocele on testicular and semen parameters.

Even though the varicocele detected in adolescents was initially considered a completely innocent finding, most researchers today agree that when varicocele starts early and continues into adulthood, the damage to germ cells is greater, as is the case with testicular atrophy. Paduch and Niedzielski [12] found that in adolescents with moderate to severe varicocele, rehabilitation reversed the cessation of testicular growth and resulted in growth within 12 months of treatment. Bedir et al. (13) showed that the reduced testicle reached its normal volume by 70% in children who underwent varicocelectomy before adolescence.

Various methods are currently available for varicocele detection, including thermography, in addition to physical examination. It can also be diagnosed by Color Doppler ultrasonography method even though physical examination is the most important detection method.

It is known in sperm analysis examinations that varicocele causes a decrease in sperm count and motility and deterioration of sperm structures (teratozoospermia) and results in infertility in patients. Sperm parameters improve by 70% and 35% pregnancy is achieved if varicocele is diagnosed early and treated surgically. Çayan, Ozbey et al. [14] reported that if spontaneous pregnancy could not be achieved in the spouses of patients who underwent varicocele surgery, the pregnancy rates increased with assisted reproductive techniques. Previous studies have declared that varicocele causes damage by increasing oxidative stress in the testicle, but recent studies have reported that oxidative stress is caused by testicular temperature increase due to varicocele [15-18].

In addition, varicocele recurrence may occur in patients undergoing varicocelectomy and the diagnosis of recurrence has high false positivity and negativity rates with existing methods. Therefore, it may be useful to measure the testicular temperature in the diagnosis of recurrent varicocele.

The testicles are located in the scrotal sac and at a temperature 3-4℃ lower than the body temperature. Infrared digital thermographic examination of the scrotum is a sensitive, non-invasive diagnostic method in the diagnosis of early varicocele through temperature measurement from the scrotal skin surface. Infrared digital thermography of the scrotum is performed using a highly sensitive infrared camera for temperature measurement in the scrotal area. Thermography is performed with the lower part of the body naked and standing. The patient should be kept at room temperature (22-23℃) for 10 minutes before the procedure. The patient’s legs should be opened to the sides and the scrotum should be free to sag. The penis is raised up towards the abdominal wall. The thermographic camera is placed on the front of the patient at a distance of approximately 40 cm. Three images are taken basally, during the valsalva and immediately after the valsalva. It is stated that semen disorder can also be detected with infrared digital thermography of the scrotum and it is also useful in determining the candidates for surgery. It has also been proven that it can be used in the diagnosis of low-grade varicocele and in the postoperative follow-up period. The relevant areas are the temperature values in the pampiniform plexus and testicles on the right and left sides. Increased heat areas in thermography are evaluated in two different ways in the presence of varicocele. Increased heat fields are seen as a limited hyperthermia to the upper part of the affected scrotum in some patients. This is called a Pattern A. A thermal distribution including the entire affected side scrotum is detected including the pampiniform plexus and testicle in some patients. This is called a Pattern B. Bilateral scrotal hypertension is rare, if any, it is a sign of advanced varicocele. Pattern B is an important finding for early intervention. Because the risk of testicular dysfunction is high in these patients (19-22). It was stated in the study of Merla et al. in 2004 that the temperature measured in the pampiniform plexus should be ≥34℃ and the temperature measured in the testicle on that side should be >32℃ for the diagnosis of varicocele in thermographic measurements (23). These results were supported in a more recent study. The temperature measured in the left pampiniform plexus was ≥34℃ in 83% of patients with varicocele, and the temperature measured in the left testicle was >32℃ in 92% of these patients in thermographic measurements. The authors stated that the main thermographic finding for the diagnosis of varicocele was that the temperature in the left pampiniform plexus was >34℃, and that the temperature in the left testicle was >32℃ was an indicator of varicocele. Heat difference between two pampiniform plexus was also evaluated for varicocele diagnosis in this study. If the temperature difference between the two pampiniform plexus is ≥0.6℃, it is considered as a secondary thermographic finding.

It is an objective and short-term diagnostic method; however, it has not been widely used in the past. Thermographic devices are expensive, large size and in low resolution. There is also no computer software for interpretation of images. The lack of definitive diagnostic criteria and high cost are considered to be the main reasons for this (24).

### REFERENCES

1. Nöske HD, Weidner W. Varicocele: a historical perspective. World J Urol 1999;17:151-7.

2. Shiraishi K, Matsuyama H, Takihara H. Pathophysiology of varicocele in male infertility in the era of assisted reproductive technology. Int J Urol 2012;19:538-50.

3. Andò S, Giacchetto C, Colpi G, Beraldi E, Panno ML, Lombardi A, Sposato G. Physiopathologic aspects of Leydig cell function in varicocele patients. J Androl 1984;5:163-69.

4. Dabaja A, Wosnitzer M, Goldstein M. Varicocele and hypogonadism. Curr Urol Rep 2013;14:309-14.

5. Fujisawa M, Yoshida S, Kojima K, Kamidono S. Biochemical changes in testicular varicocele. Arch Androl 1989;22:149-59.

6. Masson P, Brannigan RE. The varicocele. Urol Clin North Am 2014;41:129-44.

7. Choi WS, Kim SW. Current issues in varicocele management: a review. World J Mens Health 2013;31:12-20.

8. Dubin L, Amelar RD. Varicocele size and results of varicocelectomy in selected subfertile men with varicocele. Fertil Steril 1970;21:606- 9.

9. De los Reyes T, Locke J, Afshar K. Varicoceles in the pediatric population: Diagnosis, treatment, and outcomes. Can Urol Assoc J 2017;11:34-9.

10. Kim YS, Kim SK, Cho IC, Min SK. Efficacy of scrotal Doppler ultrasonography with the Valsalva maneuver, standing position, and resting-Valsalva ratio for varicocele diagnosis. Korean J Urol 2015;56:144-9.

11. Cina A, Minnetti M, Pirronti T, Vittoria Spampinato M, Canade A, Oliva G, et al. Sonographic quantitative evaluation of scrotal veins in healthy subjects: normative values and implications for the diagnosis of varicocele. Eur Urol 2006;50:345-50.

12. D. Paduch, J. Niedzielski, Repair versus observation in adolescent varicocele: a prospective study, J Urol, 158 (1997), pp. 1128-1132.

13. Bedir F, Keskin E, Karabakan M, Karabulut i, Yilmazel FK, Ozbey EG, Aksoy Y, Özbey I. Evaluation of testicular catch-up growth in adolescent microsurgical varicocelectomy. Turk J Urol. 2017 Jun;43(2):135-140. doi: 10.5152/tud.2017.51436. Epub 2017 May 3.

14. Cayan S, Erdemir F, Ozbey l, Turek PJ, Kadioğlu A, Tellaloğlu. Can varicocelectomy significantly change the way couples use assisted reproductive technologies? J Urol. 2002 Apr;167(4):1749-52. doi: 10.1016/s0022-5347(05)65192-0.

15. Aksoy Y, Ozbey l, Aksoy H, Polat O, Akcay F.Aksoy Y. Seminal plasma nitric oxide concentration in oligo- and/or asthenozoospermic subjects with/without varicocele Arch Androl. 2002 May-Jun;48(3):181-5. doi: 10.1080/01485010252869261.

16. Aksoy H, Aksoy Y, Ozbey l, Altuntas l, Akgay F.Aksoy. The relationship between varicocele and semen nitric oxide concentrations Urol Res. 2000 Dec;28(6):357-9. doi: 10.1007/s002400000132.

17. Hassanin AM, Ahmed HH, Kaddah AN. A global view of the pathophysiology of varicocele Andrology. 2018 Sep;6(5):654-661. doi: 10.1111/andr.12511. Epub 2018 Jul 6.

18. Shiraishi K, Takihara H, Matsuyama H. Elevated scrotal temperature, but not varicocele grade, reflects testicular oxidative stress-mediated apoptosis. World Journal of Urology volume 28, pages359-364(2010).

19. Mieusset R, Bujan L. Testicular heating and its possible contributions to male infertility: a review. Int J Androl 1995;18:169-84.

20. Kulis T, Kolaric D, Karlovic K, Knezevic M, Antonini S, Kastelan Z. Scrotal infrared digital thermography in assessment of varicocele - pilot study to assess diagnostic criteria. Andrologia 2012;44:780- 5.

21. Trum JW, Gubler FM, Laan R, van der Veen F. The value of palpation, varicoscreen contact thermography and colour Doppler ultrasound in the diagnosis of varicocele. Hum Reprod 1996;11:1232-5.

22. Gat Y, Zukerman ZVI, Bachar GN, Feldberg DOV, Gornish M. Adolescent varicocele: is it a unilateral disease? Urology 2003;62:742-6.

23. Merla A, Ledda A, Donato LD, Romani GL. Assessment of the effects of varicocelectomy on the thermoregulatory control of the scrotum. Fertil Steril 2004;81:471-2.

24. Kulis T, Knezevic M, Karlovic K, Kolaric D, Antonini S, Kastelan Z. Infrared digital thermography of scrotum in early selection of progressive varicocele. Med Hypotheses 2013;81:544-6.

As a result, due to above-mentioned drawbacks and the insufficiency of present solutions in the art, it is necessary to make an improvement in the related technical field.

### OBJECT OF THE INVENTION

The invention aims to provide a structure having different technical features that are novel in this field, different from the embodiments used in the present art.

The primary object of the invention is to develop a system that will give a real-time warning when the testicular temperature of varicocele patients increases. The user will be warned when the testicular temperature is ≥34° C.

An object of the invention is to provide real-time follow-up by measuring testicular temperature with smart underwear and by diagnosing primary and especially recurrent varicocele with an increase in temperature.

An object of the invention is to provide a smart underwear that can be converted into suspensory underwear when necessary and does not need to wear a second suspensory underwear for both preoperative and postoperative suspensory underwear for varicocele patients.

False positivity and negativity rates are quite high in the diagnosis of recurrent varicocele, and the diagnosis of recurrent varicocele can be easily made with the smart underwear subject to the invention and unnecessary second surgeries are prevented with the current diagnostic methods. The recurrence will be detected according to varicocele, temperature values and testicular movement.

The invention comprises a smart underwear system for detecting varicocele disease and monitoring varicocele patients in order to achieve the objects described above, characterized by comprising:
at least 2 temperature sensors (5) that enable the temperature of the right and left testicles to be measured,
microcontroller (2),
   for evaluating the data received from the temperature sensors (5) and, when a certain temperature value is exceeded, warning the user to bring his testicles to the appropriate temperature by means of the vibration motor (3),
   providing information to the user and/or doctor via SMS and e-mail when the testicular temperature exceeds the specified temperature,
   transferring and monitoring temperature and disease-related data to the cloud service.

The structural and characteristic features of the present invention will be understood clearly by the following drawings and the detailed description made with reference to these drawings and therefore the evaluation shall be made by taking these figures and the detailed description into consideration.

### FIGURES FOR UNDERSTANDING OF THE INVENTION

FIG. 1 is a representational illustration of the smart underwear.

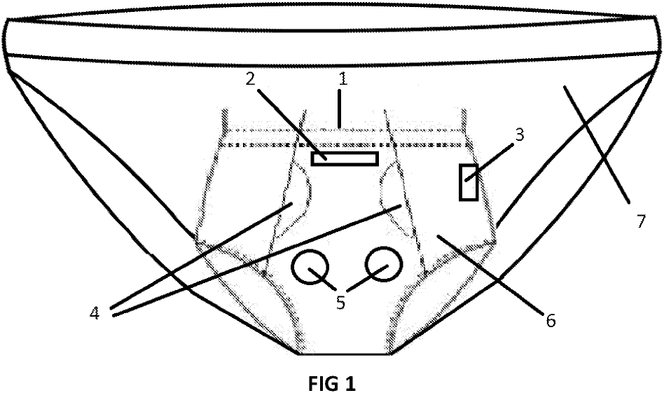

The drawings are not necessarily drawn to scale and details which are not necessary for the understanding of the present invention may be omitted. In addition, elements that are substantially identical or have substantially identical functions are denoted by the same reference signs.

### LIST OF THE REFERENCE NUMBERS

1. Servo motor
2. Microcontroller
3. Vibration motor
4. Motion Sensor
5. Temperature Sensor
6. Suspensory Underwear
7. Underwear

### DETAILED DESCRIPTION OF THE INVENTION

In this detailed description, the preferred embodiments of the invention are merely described for a better understanding of the subject matter and without any limiting effect.

The invention relates to the smart underwear (7) system used in the field of urology in the healthcare industry for the detection of varicocele disease and for the follow-up of varicocele patients.

There are 2 temperature sensors (5) and 2 motion sensors (4), 1 vibration motor (3), 1 servo motor (1), microcontroller (2) and suspensory underwear (6) on the smart underwear (7) subject to the invention.

There are at least 2 temperature sensors (5) and they allow the temperature of the right and left testicles to be measured. The microcontroller (2) evaluates the data received from the temperature sensors (5) and, when a certain temperature value is exceeded, it is stimulated by the vibration motor (3) to bring the user’s testicles to the appropriate temperature. The user is also informed via SMS and e-mail when the testicular temperature exceeds the specified temperature. In addition, SMS and e-mails are also sent to the doctor in real time via microcontroller (2). The data about the temperature is transferred to the cloud service through the microcontroller (2), the doctor and the patient can see the values on the web and examine the graphics. The doctor can observe the change in temperature values in people diagnosed with or suspected of varicocele in this way. In addition, doctors and patients can follow this data through a mobile application.

The smart underwear (7) of the invention can also be transformed into suspensory underwear (6) that provide protection of the testicles. Transform of smart underwear (7) into suspensory underwear (6) is carried out by the microcontroller (2). The smart underwear (7) will be converted into the suspensory underwear (6) via the servo motor (1) with the command sent by the microcontroller (2). The servo motor (1) moves the encoder, shaft, gear, and motor in it and rotates the shaft as an angular value. The rotation direction, speed and rotation angle of the servo motor (1) will be determined and can be transformed into suspensory underwear (6) suitable for the user. The motion sensor (4) will determine the position of the testicles and ensure the conversion of the most appropriate suspensory underwear (6). The suspensory underwear (6) is transformed after the appropriate conditions for both testicles are determined by the motion sensor (4). Motion sensors (4) transmit the right and left testicle movements to the microcontroller (2) and the most appropriate suspensory underwear (6) position is determined. The conversion of the motion sensors (4) and the suspensory underwear (6) is monitored, and any friction or possible problems will be minimized through the microcontroller (2). In addition, position and testicular movements are monitored and transferred to the cloud service through the microcontroller (2) after the motion sensors (4) are transformed into the suspensory underwear (6). It is possible to see what kind of changes occur instantly after using the suspensory underwear (6) transforms and suspensory underwear (6) on the mobile application.

1. A smart underwear (7) system that enables the detection of varicocele disease and the follow-up of varicocele patients, characterized by comprising:
at least 2 temperature sensors (5) that enable the temperature of the right and left testicles to be measured,
microcontroller (2),
for evaluating the data received from the temperature sensors (5) and, when a certain temperature value is exceeded, warning the user to bring his testicles to the appropriate temperature by means of the vibration motor (3),
providing information to the user and/or doctor via SMS and e-mail when the testicular temperature exceeds the specified temperature,
transferring and monitoring temperature and disease-related data to the cloud service.

2. The smart underwear (7) system according to claim 1, characterized by comprising a mobile application that allows the patient and the doctor to track the data transferred to the cloud service.

3. The smart underwear (7) system according to claim 1, characterized by comprising a servo motor (1) that provides the transform of smart underwear (7) into suspensory underwear (6) that ensure the protection of the testicles via the command sent by the microcontroller (2).

4. The smart underwear (7) system according to claim 1, characterized by comprising motion sensors (4) that enable the position of the testicles to be determined and enable the smart underwear (7) to be converted into suspensory underwear (6) in a position suitable for the testicles by transmitting the right and left testicle movements to the microcontroller (2).
